(19) United States
(12) Patent Application Publication
(10) Pub. No.: US 2016/0106575 A1
(43) Pub. Date: Apr. 21, 2016

(54) Thermo-Cyro Hand Therapy Device

(71) Applicant: Robert Ffrench, New Minas (CA)

(72) Inventor: Robert Ffrench, New Minas (CA)

(21) Appl. No.: 14/517,953

(22) Filed: Oct. 20, 2014

(51) Int. Cl. A61F 7/02

(52) U.S. Cl. CPC A61F 7/02; A61F 2007/0036

(57) ABSTRACT

I have invented a new process for the treatment of movement limitations caused by arthritis, strains and sprains to the hand(s) of the wearer. The invention is a therapy device for contacting and applying hot or cold therapy to the treatment area of a user (hand). The shape will enable the invention to conform to the shape of the wearer's hand in a natural fashion. The device will be filled with encapsulated phase change material of a type which will provide a uniform distribution of phase change material inside the device, resulting in balanced temperature distribution for hot or cold therapy.

The device being rectangular in shape with rounded corners and has four holes in descending sizes cut away in it, which receive the fingers and allow placement at the base of the fingers.

The device comprises a soft flexible envelope of a polyurethane material, which receives the fingers and allows effective placement of the device for continued use of the hand while undergoing therapeutic treatment.

1. A therapy device for contacting and applying hot or cold therapy to a hand/treatment area of a user, the rectangular shape of the device will enable the user to receive pain relief and support to the hand.

2. A therapy device for contacting and applying hot or cold therapy to a treatment area (hand) of a user, comprising of four holes which allow for insertion of the fingers and enable direct contact to the affected treatment areas which require the application of hot or cold therapy respectively.

3. A therapy device for contacting and applying hot or cold therapy to a treatment area (hand) of a user, comprising a soft, flexible, polyurethane sealed envelope and filled with a phase change material within said envelope.
